(19) United States
(12) Patent Application Publication
(10) Pub. No.: US 2005/0171206 A1
(43) Pub. Date: Aug. 4, 2005

(54) Therapeutical use

(76) Inventors: Christina Brahe, Rome (IT); Giovanni Neri, Rome (IT); Francesco Danilo Tiziano, Rome (IT)

Correspondence Address: MCDERMOTT WILL & EMERY LLP;Suite 700, 4370 La Jolla Village Drive, San Diego, CA, 92122, US

(21) Appl. No.: 10/971,527

(22) Filed: Oct. 21, 2004

(30) Foreign Application Priority Data
Jan. 30, 2004 (SE) 0400184-8

(51) Int. Cl. A61K 31/198; A61K 31/192

(52) U.S. Cl. 514/570; 514/565

(57) ABSTRACT

The invention relates to a method for the treatment of spinal muscular atrophy comprising administering a therapeutically effective amount of a therapeutically acceptable salt of phenylbutyrate to a subject in need of treatment of spinal muscular atrophy.

### FIELD OF THE INVENTION

The present invention relates to a method for the treatment of spinal muscular atrophy comprising administering a therapeutically effective amount of a therapeutically acceptable salt of phenylbutyrate to a subject in need of treatment of spinal muscular atrophy.

### BACKGROUND OF THE INVENTION

All literature and patent references in this description are explicitly incorporated herein by reference in their entirety.

Proximal spinal muscular atrophy (SMA) is a clinically heterogeneous group of neuromuscular disorders characterized by degeneration of the anterior horn cells of the spinal cord. Patients suffer from symmetrical weakness of trunk and limb muscles, the legs being more affected than the arms and the proximal muscles weaker than the distal ones; diaphragm, facial and ocular muscles are spared. According to the International Consortium on SMA, three forms of childhood-onset SMA (types I, II and III) can be distinguished on the basis of age of onset and severity of the clinical course assessed by clinical examination, muscle biopsy and electromyography (EMG) (Munsat T L, Davies K E (1992). Meeting Report. International SMA Consortium meeting. Neuromusc Disord 2:423-428. AA.VV. Miology. Basic and clinical—2^{nd} ed.—McGraw Hill Inc. Vol. II-P. 1837-1853 AA.VW. (1984) The International Review of Child Neurology: Progressive Spinal Muscular Atrophies. Raven Press, p. 55-91. Russman B S, Iannacone S T, Buncher C R, Samaha F J White M, Perkins B, Zimmerman L, Smith C, Burhans K, Barker L (1992) J Child Neurol 7:347-353. Zerres K, Rudnik-Schoneborn S (1995) Arch Neurol 52:518-523. Russman B S, Iannacone S T, Buncher C R, Samaha F J White M, Perkins B, Zimmerman L, Smith C, Burhans K, Barker L (1992) J Child Neurol 7:347-353).

Type I (Werdnig-Hoffmann disease) is the most acute and severe form, with onset before six months and death usually before two years; children are never able to sit without support. Symptoms of the disease can be present in utero, as reduction of foetal movements, at birth, or appear more often within the first four months of life. Children affected are particularly floppy with feeding difficulties and diaphragmatic breathing. Death is generally due to respiratory insufficiency.

Type II (intermediate, chronic form) has onset between six and eighteen months of age; muscular fasciculations are common, and tendon reflexes progressively reduce. Children are unable to stand or walk without aid. Most of patients generally develop a progressive muscular scoliosis which can require surgical correction through arthrodesis. Life expectancy is generally reduced and quality of life is severely compromised.

Type III (Kugelberg-Welander disease) is a mild, chronic form, with onset after the age of 18 months; motor milestones achievement is normal, and deambulation can be preserved until variable ages. Life expectancy is almost normal but quality of life is markedly compromised.

From a genetic point of view, SMA is an autosomal recessive condition, caused by disruption of SMN1 gene, located in 5q13 (Lefebvre S., Burglen L., Reboullet S., Clermont O., Burlet P., Viollet L., Benichou B., Cruaud C., Millasseau P., Zeviani M., Le Paslier D., Frezal J., Cohen D., Weissenbach J., Munnich A., Melki J. (1995). Cell 80: 155-165). This gene is absent in the majority of patients (95%), and small intragenic mutations have been described in 2-3% of cases. The incidence of the disease varies from 1/6000 to 1/10000, being healthy carriers quite common (1/40-1/50) in general population (Wirth B., Schmidt T., Hahnen E., Rudnik-Schoneborn S., Krawczak M., Muller-Myhsok B., Schonling J., Zerres K. (1997). Am. J. Hum. Genet. 61: 1102-1111.).

All patients have at least one, generally two to four, copies of the SMN2 gene which is nearly identical to SMN1, and encodes the same protein. However, the SMN2 genes produce only low levels of full-length SMN protein. The clinical severity of SMA patients inversely correlates with the number of SMN2 genes and with the level of functional SMN protein produced (Lorson C L, Hahnen E, Androphy E J, Wirth B. Proc Natl Acad Sci 1999; 96:6307-6311. Vitali T, Sossi V, Tiziano F, et al. Hum Mol Genet 1999; 8:2525-2532. Brahe C. Neuromusc. Disord. 2000; 10:274-275. Feldkotter M, Schwarzer V, Wirth R, Wienker T I, Wirth B. Am J Hum Genet 2002; 70:358-368. Lefebvre S, Burlet P, Liu Q, et al. Nature Genet 1997; 16:265-269. Coovert D D, Le T T, McAndrew P E, et al. Hum Mol Genet 1997; 6:1205-1214. Patrizi A L, Tiziano F, Zappata S, Donati A, Neri G, Brahe C. Eur J Hum Genet 1999; 7:301-309.)

The mechanism leading to motorneuron loss and to muscular atrophy still remains obscure, although the availability of animal models of the disease is rapidly increasing knowledge in this field (Frugier T, Tiziano F D, Cifuentes-Diaz C, Miniou P, Roblot N, Dierich A, Le Meur M, Melki J. (2000) Hum Mol Genet. 9:849-58; Monani U R, Sendtner M, Coovert D D, Parsons D W, Andreassi C, Le T T, Jablonka S, Schrank B, Rossol W, Prior T W, Morris G E, Burghes A H. (2000) Hum Mol Genet 9:333-9; Hsieh-Li H M, Chang J G, Jong Y J, Wu M H, Wang N M, Tsai C H, Li H. (2000) Nat Genet 24:66-70; Jablonka S, Schrank B, Kralewski M, Rossoll W, Sendtner M. (2000) Hum Mol Genet. 9:341-6). Also the function of SMN protein is still partially unknown, and a great bulk of studies indicates that it can be involved in mRNA metabolism (Meister G, Eggert C, Fischer U. (2002). Trends Cell Biol. 12:472-8; Pellizzoni L, Yong J, Dreyfuss G. (2002). Science. 298:1775-9), and probably in transport of proteins/mRNA to neuromuscular junctions (Ci-fuentes-Diaz C, Nicole S, Velasco M E, Borra-Cebrian C, Panozzo C, Frugier T, Millet G, Roblot N, Joshi V, Melki J. (2002) Hum Mol Genet. 11:1439-47; Chan Y B, Miguel-Aliaga I, Franks C, Thomas N, Trulzsch B, Sattelle D B, Davies K E, van den Heuvel M. (2003) Hum Mol Genet. 12:1367-76; McWhorter M L, Monani U R, Burghes A H, Beattie C E. (2003) J Cell Biol. 162:919-31; Rossoll W, Jablonka S, Andreassi C, Kroning A K, Karle K, Monani U R, Sendtner M. (2003) J Cell Biol. 163:801-812).

Sodium 4-phenylbutyrate (triButyrate®) has been used in clinical trials on sickle cell anemia and beta-thalassemia (Collins A F, Pearson A. Giardina P et al. Blood 85:43-49.). Furthermore, triButyrate® has been used for urea cycle disorders treatment for more than a decade with a significant improvement of mental performance of patients and few toxic effects (Batshaw M L, MacArthur R B, Tuchman M. (2001) J Pediatr. 38(1 Suppl):S46-54).

Brahe and coll. have provided evidence that 4-phenylbutyric acid (PBA), is effective in increasing the expression of the SMN2 genes. PBA was found also effective in enhancing SMN protein levels and the number of SMN containing nuclear structures (Andreassi C, Angelozzi C, Tiziano F D, Vitali T, De Vincenzi E, Boninsegna A, Villanova M, Bertini E, Pini A, Neri G, Brahe C. Eur. J. Hum. Genet. 2004; 12:59-65). However, this is an in vitro study on cell cultures and thus no conclusions regarding the clinical effect can be inferred. Phenylbutyric acid cannot be used for the treatment of patients, because the smell and taste is unacceptable. Phenylbutyric acid metabolises mainly into butyric acid.

No cure for SMA is available to date. In the past there have been attempts to treat SMA patients. Three recent clinical trials for SMA patients are as follows:
a) The drug gabapentin was used in one study (Miller R G, Moore D H, Dronsky V, Bradley W, Barohn R, Bryan W, Prior T W, Gelinas D F, Iannaccone S, Kissel J, Leshner R, Mendell J, Mendoza M, Russman B, Samaha F, Smith S; SMA Study Group. (2001) J. Neurol. Sci. 91:127-31). However the drug gabapentin did not show improvement of the symptoms.
b) The drug riluzole was used in a study of very few patients with SMA I. (Russman B S, Iannaccone S T, Samaha F J (2003) Arch Neurol 60:1601-3). In this study no statistically significant improvement was proved.
c) The drug albuterol was used in a clinical study of SMA patients. This drug leads to an improvement in clinical symptoms, but it has an aspecific effect on muscle strength. There is no evidence that it has any effect on SMN-gene expression. Thus albuterol does not act directly on the molecular defect of SMA. (Kinali M, Mercuri E, Main M, De Biasia F, Karatza A, Higgins R, Banks L M, Manzur A Y, Muntoni F (2002) Neurology 59:609-10)

It is an object of the present invention to provide a method for the treatment of SMA, which does not suffer from the drawbacks of the drugs mentioned above.

### BRIEF DESCRIPTION OF THE FIGURES

FIG. 1 shows the results of SMN expression studies in PB treated and untreated subjects. This study is further described in example 1.

FIG. 2 shows individual results on the Hammersmith functional scale at T0, T1 and T2. This study is further described in example 2.

FIG. 3 shows mean scores at baseline (T0), 3 weeks (T1) and 9 weeks (T2) after treatment was started in children below and above 5 years of age. This study is further described in example 2.

FIG. 4 shows the difference in score between baseline (T0) and after 9 weeks of treatment (T2) in the study group and between T0 and T3 (6 month interval) in the control group. This study is further described in example 2.

### DESCRIPTION OF THE INVENTION

The present invention provides an efficient treatment for SMA.

In an aspect of the present invention there is provided a method for treatment of spinal muscular atrophy comprising administering a therapeutically effective amount of a therapeutically acceptable salt of phenylbutyrate to a subject in need of treatment of spinal muscular atrophy.

Sodium 4-phenylbutyrate (triButyrate®) is known to inhibit the activity of histone deacetylases, thus inducing hyperacetylation of histones, with consequent upregulation of gene transcription. triButyrate® modulates the transcription of a variety of genes including foetal haemoglobin.

Recently, evidence was given that triButyrate® penetrates into the cerebrospinal fluid (CSF) after i.v. administration in non-human primates (Berg S, Serabe B, Aleksic A et al. Cancer Chemother Pharmacol 2001; 47:385-390), thus indicating that triButyrate® is able to cross the blood-brain barrier.

In example 1 and 2 triButyrate® is used. Sodium phenylbutyrate (triButyrate®) metabolises mainly into sodium phenylacetate (Piscitelli S. C. et al. (1995) Journal of Clinical Pharmacology 35: 368-373.), which in turn will be activated to other substances according to well known pathways in the body, (Stryer, Biochemistry 4^{th} ed, Chapter 24 and 25, W.H. Freeman, New York 1995). More precisely according to recent publications sodium phenylbutyrate (triButyrate®) metabolises to approximately 75% sodium phenylacetate and 25% butyrate. Some studies on cancer indicate that the effect of triButyrate® is greater than that of sodium phenylacetate, which for this indication may indicate that the butyrate metabolite has an effect as well.

Sodium phenylacetate is the active substance. The metabolic pathway for triButyrate® is different from that of PBA. PBA is degraded to butyric acid whereas triButyrate® is degraded to sodium phenylacetate. In the light of what was previously known about PBA and triButyrate® it is thus surprising that triButyrate® has the effect disclosed in this description.

The inventors have found that a salt of phenylbutyrate is effective for the treatment of SMA. This is unexpected and can not be predicted from what is previously known.

According to the present invention a therapeutically acceptable salt of 4-phenylbutyric acid is used. The salt may for instance be selected from the group consisting of salts of alkali metals and alkaline earth metals and suitable salts of all amino acids as well as ammonium salts. Specific examples of salts are salts selected from the group consisting of sodium, potassium, magnesium, calcium and arginine salts. The sodium salt is particularly preferred in the present invention.

The drug may be administered in several ways. The drug may be administered intravenously as has been done for the same drug for patients with urea cycle disorders (Batshaw et al., J. of Pediatrics, 138:S46-S55, 2001). In a particularly preferred embodiment of the present invention the drug is administered orally. In another preferred embodiment of the present invention the drug is administered using suppository formulations. In yet another preferred embodiment of the present invention the drug is administered topically. A person skilled in the art realises that also other administration routes may be used. The active substance of the present invention is administered together with conventional excipients and carriers.

For one embodiment of the present invention a preferred dose is 450 to 600 mg/kg/d. In a particularly preferred embodiment of the present invention the dose is about 500 mg/kg/d. A person skilled in the art realises that also doses around 500 mg/kg/d are preferred, such as 510, 520, 530, 540, and 490, 480, 470, 460, 450 mg/kg/d, also values in between these mentioned values are preferred such as 491, 492, 493, 494, 495, 496, 497, 498, 499, 501, 502, 503, 504, 505, 506, 507, 508, 509 mg/kg/d. Also other doses are preferred, such as 560, 570, 580, and 590 mg/kg/d. The dose is calculated per body weight of the patient in kg. In another preferred embodiment of the present invention the dose is even higher. Doses as high as up to 40 to 50 g per day for a patient may be used. Even higher doses may also be used according to the present invention, Professor Saul Brusilow at the John Hopkins University in Baltimore, USA has used as much as 200 g per person and day, and the only noticed side-effect was increased blood pressure from the sodium ions. Doses up to 200 g per person and day or higher are also included in the present invention. A person skilled in the art realises that also other doses may work, although they are not optimal, such as doses in the interval 50 to 1500 mg/kg/d. Doses according to the present invention are for instance 50, 100, 150, 200, 250, 300, 350, 400 mg/kg/d, as well as 650, 700, 750, 800, 850, 900, 950, 1000, 1050, 1100, 1150, 1200, 1250, 1300, 1350, 1400, 1450 and 1500 mg/kg/d.

In a preferred embodiment of the present invention the drug is administered to maintain consistently a high and even level of the drug in the patient. A person skilled in the art realises how to distribute the doses over the day to achieve this. A person skilled in the art also realises how to use suitable administration systems to achieve this.

In example 1 below it is shown that oral administration of the sodium salt of phenylbutyrate (triButyrate®) significantly increases SMN2 expression in leukocytes of SMA patients. In the pilot study in example 1 performed on 6 SMA type II and III patients, SMN mRNA analysis in leukocytes showed for all patients a significant increase in relative SMN2 full length transcript levels in one or more blood samples obtained during triButyrate® administration, compared to baseline. The mean increase in patient's transcript levels ranged from 43% up to 240%. These data show that SMN2 gene expression is considerably increased by triButyrate® and suggest that the compound, owing also to its favourable pharmacological properties, could be a good candidate for the treatment of SMA.

A pilot trial in example 2 below showed a significant increase in motor function in 10 children with SMA type II, treated with triButyrate® for 9 weeks using an intermittent regimen consisting of one week drug administration and one week off. The improvement was already obvious after 2 courses of treatment (3 weeks after T0, when treatment was started) and a further improvement was noted at the end of the 5^{th} course (9 weeks after T0). The increase in functional ability reached statistical significance and was always associated with some noticeable improvement in everyday life activities.

The data in example 1 and 2 provide a strong rational for a method comprising administration of triButyrate® for the treatment of SMA patients.

### EXAMPLES

The following examples serve the purpose to further illustrate the invention and are not intended to be limiting in any way. All citations are explicitly incorporated herein in their entirety.

### Example 1

Subjects and Methods

Six SMA patients (P1-P6) and three parents (M2, M3, F6) were enrolled for a pilot trial. Four patients had SMA type II (P1-P4). P1 is a 2.5-year-old boy who had lost the ability to sit unaided. P3 is 5 years of age and P4 and P5 are both 9-year-old. Two patients (P5, 38 years and P6, 15 years) had SMA type III. The trial was approved by the Ethical Committee of the Catholic University. A written informed consent was obtained from all patients/parents. triButyrate®, the sodium salt of phenylbutyrate was administered at 500 mg/kg/d (maximum dose 19 g/d), divided in 6 doses(every 4 hours) for 7 days. Blood samples were taken from patients and parents on day 0 (T0, baseline) and on days 1-4 (T1-T4) and 7 (T7) of drug administration, and from 5 healthy untreated controls on 5 consecutive days (T0-T4). Total RNA was extracted by Trizol from leukocytes immediately after hypotonic lysis of samples.

Real-Time PCR

SMN full length (SMN-fl) transcripts were measured by realtime RT-PCR using ABI-PRISM 7700 Sequence Detector System (Applied Biosystems) as described elsewhere. (Andreassi C, Angelozzi C, Tiziano F D, Vitali T, De Vincenzi E, Boninsegna A, Villanova M, Bertini E, Pini A, Neri G, Brahe C. Eur. J. Hum. Genet. 2004; 12:59-65) Transcripts were amplified at least twice in triplicate or quadruplicate. SMN transcript levels were calculated by comparing SMN versus glyceraldehyde-3-phosphate dehydrogenase transcripts, whose expression is not affected by triButyrate®. (Andreassi C, Angelozzi C, Tiziano F D, Vitali T, De Vincenzi E, Boninsegna A, Villanova M, Bertini E, Pini A, Neri G, Brahe C. Eur. J. Hum. Genet. 2004; 12:59-65) The relative amounts of SMN-fl transcripts in samples obtained from treated patients/parents and untreated controls were normalized versus those of T0.

Myometry

Muscle strength was assessed using a hand-held dynamometer (Citec, CIT Technics BV). (Merlini L, Mazzone E S, Solari A, Morandi L. Muscle Nerve 2002; 26:64-70) Patients were tested independently four times by two raters, and the highest measure of the maximal voluntary isometric contraction was selected. Inter-rater coefficients (ICC) were determined as described by Shrout et al. (Shrout P E, Fleiss J L. Pscychol Bull 1979; 86:420-428)

Statistical Analysis

F of Fisher, ANOVA and T of Student for independent variables tests were performed to assess statistical significance by using Winstat 4.01 and SPSS 10.0.1 for Windows software. P values <0.05 were accepted as significant.

Results

SMN Expression Studies

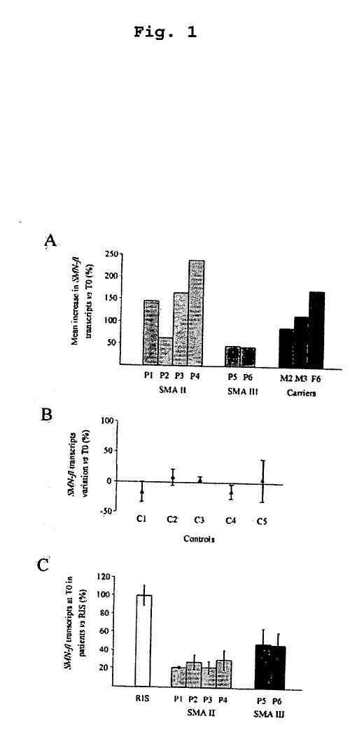

SMN mRNA analysis showed for all patients a significant increase in relative SMN-fl transcript levels in one or more blood samples obtained during triButyrate® administration compared to baseline (table 1.1, FIG. 1A). The relative amount of SMN-fl mRNA during treatment varied considerably, both among the different subjects and between the different blood samples of the same subject. The mean increase in patients transcript levels ranged from 43% for P6 up to 240% observed in P4, and from 90% to 170% in the parents. To investigate whether SMN-fl transcript levels are subject to physiologic variation, SMN expression was studied in five healthy untreated controls during five days. A slight to moderate variation in SMN-fl levels versus T0 was observed in the controls with mean variation between 4.2% and 16.5% and SD ranging from 5.1% to 34.9% (FIG. 1B). Variation in SMN-fl levels in treated patients/parents versus controls was statistically significant with three different tests. To estimate the percentage of the SMN-fl transcript levels in the patients before treatment respective to that of unaffected subjects, their baseline SMN mRNA levels were compared to a reference internal standard, calculated as average amount of SMN-fl mRNA of the controls at T0. The relative SMN-fl transcript levels were 22-28% and 47-48% of control levels in the type II and type III patients, respectively (FIG. 1C).

Clinical Observations

All subjects tolerated the drug well except for P6 who on day 2 complained of dizziness and tinnitus, which resolved immediately after reducing dosage from 18 g/d to 12 g/d. Full blood counts and liver function tests, performed for all subjects at days 0 and 7, did not show significant changes. The first patient studied (P5) reported a reduction of hand tremor at day 3 of treatment and subsequently complete absence of tremors lasting for 4 days after the end of the trial. The second patient (P1), a young child with severe type II phenotype, showed a slight improvement in head and trunk control. These subjective improvements prompted us to perform myometry in the other four patients on day 0 and 7 (table 1.2). The three type II patients (P2-4) showed an increase in leg muscle strength on day 7 compared to baseline, which was statistically significant in two (P3 and P4) and was less pronounced, but still measurable, in the other (P2). Arm strength was also increased in patients P3 and P4, but this was less obvious than the increase in leg strength. In the type III case (P6) myometry showed no changes in muscle performance.

Discussion

SMN2 gene expression can be significantly increased in SMA patients by oral administration of a drug, according to the present study. triButyrate®, a sodium salt of an aromatic fatty acid, was used and it is well tolerated by young children. Recently, evidence was given that triButyrate® can cross the blood-brain barrier. (Berg S, Serabe B, Aleksic A, et al. Cancer Chemother Pharmacol 2001; 47:385-390.) The reason for the observed variability in SMN gene expression following triButyrate® administration is unclear. Given the short half-life of triButyrate® (0.8-1 hour) fluctuations in relative SMN transcript levels in the same subject may be related to varying levels of plasmatic triButyrate® concentrations at different intervals between drug administration and blood sampling. When the extent of reduction in SMN-fl levels was investigated in the patients before treatment compared to unaffected individuals, it was found that the type II and III patients had approximately 25% and 50%, respectively, of SMN-fl levels of controls. If it is considered that in all patients a more than 100% increase in SMN-fl transcripts was detected in at least one blood sample during treatment it may be speculated that SMN-fl levels in leukocytes of SMA type II patients could transiently exceed the baseline levels of type III patients and that the latter could achieve levels similar to that of controls. The observation of an increase in muscle strength after one week of treatment is related to the increase in SMN2 gene expression. Interestingly, the highest values of mean increase in SMN transcript levels were found in the two patients who also showed the highest increase in limb megascores and no change in muscle strength could be recorded in the case who had taken triButyrate® at a 33% reduced dosage and also had the lowest mean increase in SMN-fl transcripts. In conclusion, the finding of the inventors that SMN2 transcipt levels can be elevated in SMA patients by administration of triButyrate® opens the perspective for a pharmacological treatment of SMA.

FIG. 1 shows results from SMN expression studies in triButyrate® treated and untreated subjects.
A. Percent mean increase in SMN-fl transcript levels in leukocytes of patients and carriers during one week of triButyrateo treatment.
B. Variation of SMN mRNA in healthy controls during 5 days relative to mean level at T0. Error bars indicate SD.

C. Percent of SMN-fl transcript levels in patients before treatment relative to that of unaffected individuals. Reference Internal Standard (RIS) indicates the average amount of SMN-fl mRNA in five unaffected individuals at T0. Error bars indicate SD.

TABLE 1.1
Percent SMN-fl transcripts variation in triButyrate ®
treated patients and parents versus T0

| Patients/patents | T1 | T2 | T3 | T4 | T7 |
|---|---|---|---|---|---|
| (sex) | mean(+−SD) | mean(+−SD) | mean(+−SD) | mean(+−SD) | mean(+−SD) |
| P1 (M) | +280 (0.7) | ND | −15 (0.1) | −18 (0.2) | +333 (0.5) |
| P2 (M) | +73 (0.0) | +27 (0.0) | +21 (0.1) | +129 (0.3) | ND |
| P3 (M) | +432 (0.2) | +63 (0.1) | +82 (0.2) | +190 (0.5) | +56 (0.2) |
| P4 (F) | +53 (0.1) | +184 (0.6) | +7 (0.2) | +120 (0.4) | +827 (1.9) |
| P5 (M) | −1 (0.0) | +35 (0.3) | ND | −3 (0.0) | +155 (0.1) |
| P6 (F) | +54 (0.2) | +25 (0.1) | +131 (0.5) | +21 (0.0) | −15 (0.4) |
| M2 (F) | +68 (0.0) | +14 (0.0) | +116 (0.0) | +156 (0.2) | ND |
| M3 (F) | +65 (0.1) | −2 (0.0) | −37 (0.0) | +363 (0.6) | +190 (0.2) |
| F6 (M) | +117 (1.0) | +96 (0.1) | +197 (0.7) | +387 (0.9) | +71 (0.3) |

P1-P6: patients;
M2, M3: mothers of P2 and 3, respectively;
F6: father of P6.
F of Fisher (case versus control, T1-T4): P < 0.0001;
ANOVA (patients/parents T1-T7 versus T0): P < 0.0047;
T of Student (patients/parents T1-T7 versus T0): P < 0.03 except for T2.

TABLE 1.2
Outcome of myometry

| | P2 | | P3 | | P4 | | P6 | |
|---|---|---|---|---|---|---|---|---|
| | T0 | T7 | T0 | T7 | T0 | T7 | T0 | T7 |
| Hand-grip | 18 (0.6) | 18 (0.8) | 6 (1) | 7 (0.81) | 3 (0.8) | 4 (1) | 57 (0.82) | 52 (0.82) |
| Elbow | 38 (0.86) | 47 (0.8) | 19 (0.81) | 35 (0.93) | 3 (1) | 15 (0.93) | 105 (0.77) | 86 (0.91) |
| flexion | | | | | | | | |
| Three- | 20 (1) | 23 (0.95) | 6 (0.83) | 9 (0.77) | 4 (0.8) | 2 (0.8) | 30 (0.96) | 37 (0.94) |
| point | | | | | | | | |
| pinch | | | | | | | | |
| Total arm | 76 | 88 | 31 | 41 | 10 | 21 | 192 | 175 |
| megascore | | | | | | | | |
| Knee | 6 (0.83) | 8 (0.87) | 6 (1) | 7 (0.87) | 5 (0.8) | 21 (0.85) | 21 (0.8) | |
| extension | | | | | 11 (0.72) | | | |
| Knee | 12 (0.91) | 16 (0.94) | 9 (0.82) | 16 (0.92) | 8 (0.62) | 12 (0.91) | 51 (0.86) | 55 (0.92) |
| flexion | | | | | | | | |
| Foot | 14 (0.85) | 15 (0.6) | 2 (0.6) | 9 (0.81) | 4 (0.7) | 11 (0.81) | 12 (0.75) | 11 (0.9) |
| dorsiflexion | | | | | | | | |
| Total leg | 32 | 39 | 17 | 32 | 17 | 34 | 84 | 88 |
| megascore | | | | | | | | |

Measures are expressed in Newtons; inter - rater correlation coefficients (ICC) are shown in parenthesis.
Arm and leg megascores are the sum of upper and lower limb measures, respectively. T0 = baseline;
T7 = day 7.
In bold the statistically significant value are indicated (P < 0.01), calculated using the chi2 test.

Measures are expressed in Newtons; inter- rater correlation coefficients (ICC) are shown in parenthesis. Arm and leg megascores are the sum of upper and lower limb measures, respectively. T0=baseline;

T7=day 7. In bold the statistically significant value are indicated (P<0.001), calculated using the chi2 test.

### Example 2

Patients and Methods:

Thirteen patients with SMA II, all with homozygous absence of SMN1, followed at the Bambino Gesu Hospital or at the UILDM centre in Rome were asked to participate in the present prospective open trial. In order to have a relatively homogeneous cohort of patients who could all be tested on the same scale only children with SMA II between 30 months and 12 years were included. Children younger than 30 months were excluded as the Hammersmith scale can only be reliably and consistently performed and scored after this age. (Main M, Kairon H, Mercuri E, Muntoni F. Eur J Paediatr Neurol. 2003;7:155-59.) Children older than 12 were excluded as after this age several complications, such as severe scoliosis and contractures are more frequent. Children who had been part of other pharmacological trials (e.g. salbutamol, creatine) in the year before the present trial started or who had had corrective surgery for scoliosis were also excluded.

The study was given approval by the Ethics Committee at the Catholic University in Rome, and informed consent was obtained from all subjects after verbal and written explanation of the trial.

The sodium salt of phenylbutyrate (triButyrate®) was administered orally in powder or tablets at 500 mg/kg/d (maximum dose 19 g/d), divided in 5 doses (every 4 hours except for an 8 hours night interval). An intermittent schedule (7 days on and 7 days off) was arbitrarily chosen, similarly to a previously reported schedule for treatment of sickle cell disease. (Atweh G F, Sutton M, Nassif I, Boosalis V, Dover G J, Wallen-stein S, Wright E, McMahon L, Stamatoyannopoulos G, Faller D V, Perrine S P Blood.1999;93:1790-97.) When this dosage was tolerated, patients were advised to continue for 9 weeks (5 weeks on and 4 weeks off).

Each patient had a baseline assessment soon before starting the medication and at 3 and 9 weeks after treatment was started, therefore at the end of 2 and 5 weeks of drug administration. The assessment consisted of a detailed clinical physiotherapy evaluation including the Hammersmith functional motor scale and, in children older than 5 years, assessment of muscle strength and of forced vital capacity.

Functional Ability

The scale consists of 20 items, each scored on a 3 point scoring system, (2 for unaided, 1 for assistance and 0 for inability). A total score can be achieved by summing the scores for all the individual items. The score can range from 0, if all the activities are failed, to 40, if all the activities are achieved.

All items have to be tested without thoracic braces or leg orthoses. The scale can be completed in a maximum of 15 minutes. (Main M, Kairon H, Mercuri E, Muntoni F. Eur J Paediatr Neurol. 2003;7:155-59.)

The scale was performed by 2 examiners (MP, SM) and each child was always assessed by the same examiner throughout the 3 assessments. Both observers had training sessions with a senior physiotherapist (MM) and the intra and inter-rater reliability was formally assessed in 5 children before starting the trial. In agreement with the previously reported data on intra and inter-observer reliability, these were >95%. (Main M, Kairon H, Mercuri E, Muntoni F. Eur J Paediatr Neurol. 2003;7:155-59.)

The data obtained in the study group were compared to those obtained in 19 untreated children with SMA. Control children were retrospectively selected from the population of children with SMA followed at the Hammersmith Hospital who are routinely assessed at 6-month intervals with the Hammersmith scale. Control children were only selected according to their age and functional ability. For each child in the study group the inventors tried to identify two controls who had the same age (±2 months) and functional score (±1 point) at T0. If more than 2 controls were available for a patient in the study group, the 2 controls who were closer in terms of age and functional ability were chosen. Two matched controls were available for all cases but one for whom only one control could be identified. All the children in the control group had assessment at the same age of the study group (T0) and after 6 months (T3). Exclusion criteria were the same as in the study group.

Myometry

The maximal voluntary isometric contraction was measured using a hand held electronic myometer (Citec, Cit techniques BV, The Netherlands) on 5 muscle groups (elbow flexion, hand grip, three point pinch, knee flexion and extension), according to the criteria suggested by Merlini et al. (Merlini L, Mazzone E S, Solari A, Morandi L. Muscle Nerve 2002;26: 64-70.) For each muscle group considered, the score (in Newtons) was obtained by totalling the best of three readings obtained on the patient's strongest side. Measurements lasted 3 to 5 seconds. The scores were grouped in a leg mega score and arm megascore by adding the best readings for each individual muscle within the subgroup and subdividing this score by the number of muscles examined. Intra-observer variation performed before the implementation of the study was less than 5%. Both myometry and measurement of joint contractures were performed by the same physiotherapist.

Forced Vital Capacity

This was measured using a standard spirometer (Spyroanalyser ST-95, Fukuda Sangyo Co, LTD, Philippines) and using the best of three attempts.

Statistical Analysis:

The primary measure of efficacy in this trial was the change from baseline to 3 and 9 weeks in motor function as determined by the Hammersmith motor scale. Secondary measures included the change from baseline to 3 and 9 weeks in myometry scores and in FVC in the children older than 5 years. The primary outcome was analysed using repeated measures ANOVA. Statistical significance was determined at p<0.05. An estimate of SD of change in each of the measures considered is presented with 95% confidence intervals. The number of children older than 5 years who could be reliably assessed for myometry and FVC was too small to allow any meaningful statistical analysis for these measures.

Results:

Thirteen of the 14 patients asked, agreed to participate in the present study. One of the 13 refused to take the tablets after the first dose as she did not like the taste. Another child developed a skin rash soon after the second week of treatment was started and therefore treatment was discontinued. A third one (aged 30 months) refused to collaborate at the time of the first assessment. The remaining ten all completed the 9 weeks schedule (4 M, 6 F). Their age ranged between 2.6-12.7 with a mean age of 6.01.

Table 2.1 shows the range, mean and SD of the different aspects of functional ability, myometry and FVC at baseline (T0), and after 3 and 9 weeks (T1 and T2).

Hammersmith Functional Scale

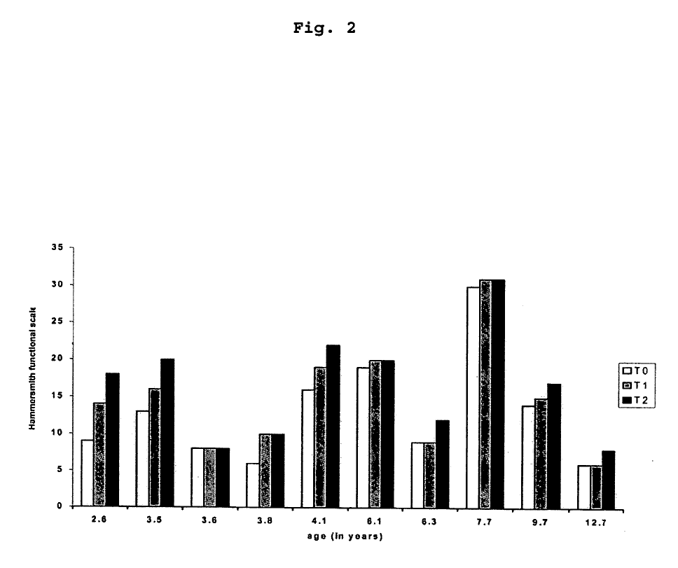

The scale was easily completed in all 10 children. The scores ranged between 6 and 30 (mean 13) at T0, between 6 and 31 (mean 14.8) at T1 and between 8 and 31 (mean 17) at T2. FIG. 2 shows individual details of the scores.

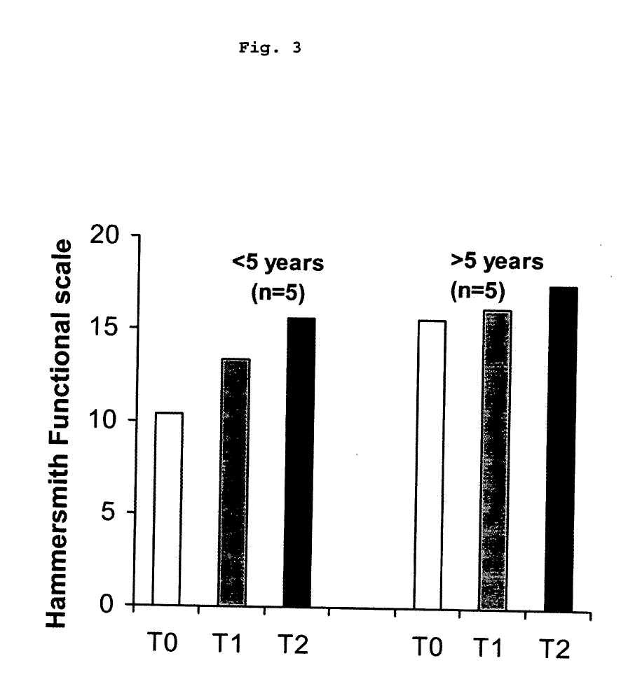

There was a significant difference between T0 and T1 (p=0.012), between T1 and T2 (p=0.008) and between T0 and T2 (p=0.004). Details of mean scores and of statistical analysis are given in table 2:1. The difference was more marked in the children younger than 5 years (FIG. 3).

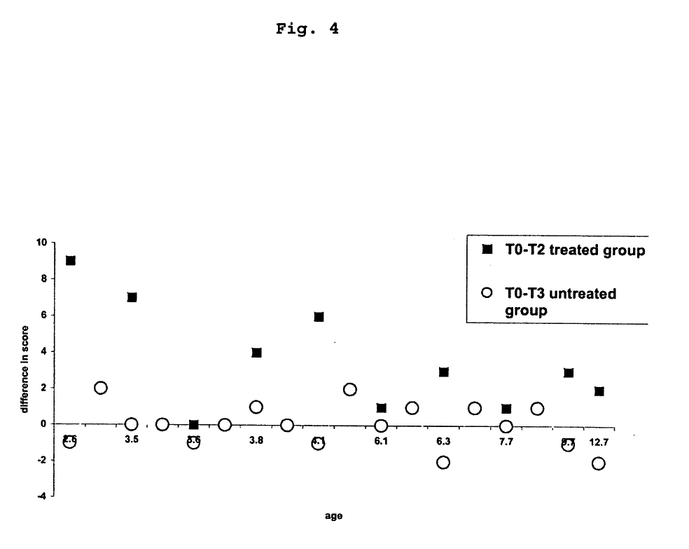

In the control group the scores ranged between 6 and 29 (mean 12.6) both at T0 and 6 months later (T3). Although there were some individual variations in the 19 untreated SMA children the overall mean at T0 and T3 was similar. (FIG. 4)

Myometry

Myometry was obtained in 4 of the 5 patients older than 5 years but could not be reliably tested in one child with severe contractures. There was a clear increase in 3 of the 4 (table 2.2).

Forced Vital Capacity

Forced vital capacity was obtained in 4 of the 5 patients in the study. There was a clear increase in 3 of the 4 (see table 2.2).

Contractures

There was no difference in major joint's ranges (hips, knees, hamstrings, tendon Achilles and elbows) documented between baseline and 9 weeks.

Side Effects

Sodium salt of phenylbutyrate was generally well tolerated by all individuals during the study with the exception of a girl who developed a skin rash after the second week of treatment started and in whom treatment was therefore discontinued. The remaining 10 all completed the trial. Three very young children complained of stomach ache after the second dose but this did not recur. Eight of the 10 children who completed the trial found the taste of the drug very unpleasant and seven of them also reported an unpleasant odour when sweating but none of them discontinued the therapy because of this. All patients would have elected to remain on treatment at the end of the trial, as they overall felt a positive effect with regard to muscle endurance.

Discussion

Despite the advances in the understanding of the pathogenesis of SMA, so far there is no effective cure. A few studies have recently suggested a possible beneficial effect of drugs such as beta-adrenergic agonist (Kinali M, Mercuri E, Main M, De Biasia F, Karatza A, Higgins B, Banks L, Manzur A Y, Muntoni F. Neurology 2000;59:609-10.) or GABA-vinyl gaba. The results of this study however showed a great variability of response. It is therefore hoped that better results could be obtained by using pharmacological agents that could modify SMN gene expression.

The inventors have recently shown that phenylbutyrate (triButyrate®), similarly to sodium butyrate can increase SMN2 gene expression in vitro. In this example the inventors used sodium phenylbutyrate rather than sodium butyrate as sodium phenylbutyrate has a relatively longer half-life (0.8-1 hour) and is better tolerated when given in relatively high doses, as previously reported in studies describing the effect of triButyrate® in children with urea disorders. (Moyer B D, Loffing-Cueni D, Loffing J, Reynolds D, Stanton B A. Butyrate increases apical membrane CFTR but reduces choride secretion in MDCK cells. Am J Physiol 1999;277:F271-6)

The present results showed a significant increase in motor function in the treated children. The improvement was already obvious after 2 courses of treatment (3 weeks after T0, when treatment was started) and a further improvement was noted at the end of the 5^{th} course (9 weeks after T0). The increase in functional ability reached statistical significance and was always associated with some noticeable improvement in everyday life activities. These results cannot be simply due to a better collaboration or to practice, as the functional scores in untreated SMA controls, matched for age and functional ability, did not significantly change within 6 months. While nine out of ten children in the study group had an increase in functional scores, which was of more than 2 points in 6 of them, the mean scores obtained at T0 and 6 months later in the control group were similar and only six of the 18 controls showed an increase in their scores which was of a single point in 4 of the 6. The small changes observed in the present control group are in agreement with previous studies describing the natural history of SMA. These studies report that SMA is a relatively stable condition suggesting that one should not expect any significant variation over a period of 2 to 6 months. (Zerres K, Rudnick-Schoneborn S Arch Neurol 1995;52:518-23.)

The magnitude of improvement in the scores in the present study group was quite variable and children below 5 years had a more obvious improvement than the older ones. This might be due to the fact that younger children tend to have less secondary complications such as contractures or scoliosis, which might affect the response to treatment.

The use of a functional scale assessing gross motor function as the primary measure of a trial is often object of controversy. On the other hand, a few studies have recently suggested that in weak children, such as those affected by SMA, gross motor function measures are more reliable than quantitative tests, such as myometry (Iannacone ST and AmSMART Group. Arch Neurol 2002;59:1445-50., Iannacone S T, Hynan L S and AmSMART Group. Arch Neurol 2003;60:1130-36.) Furthermore, this scale can be reliably used after the age of 30 months while other tools such as myometry or MRC scale cannot be easily and reliably performed before the age of 5 years. It is of interest that in the older children, in whom myometry and forced vital capacity could be measured, the results in both tests showed a similar trend to those found by using the Hammersmith scale.

Although the previous observations in vitro provide an explanation of the possible mechanism underlying this improvement, it is surprising that such a significant clinical improvement could already be noted after 3 weeks. In conclusion, the results of the present open trial suggest that phenylbutyrate given intermittently for 9 weeks can produce an improvement in SMA II patients without any significant adverse effect.

TABLE 2.1
Mean, standard deviation, standard error and confidence
interval at baseline (T0), 3 weeks (T1) and 9 weeks
(T2) after treatment was started.

| | | | Standard | 95% CI(lower | 95% CI(upper |
|---|---|---|---|---|---|
| | Mean | SD | error | bound) | bound) |
| T0 | 13 | 7.38 | 2.333 | 7.722 | 18.278 |
| T1 | 14.8 | 7.38 | 2.332 | 9.524 | 20.076 |
| T2 | 16.6 | 7.26 | 2.296 | 11.406 | 21.794 |

TABLE 2.2
Details of myometry and forced vital capacity
(FVC) at baseline (T0), 3 weeks (T1) and 9 weeks (T2) after
treatment was started.

| | myometry | | | FVC | | |
|---|---|---|---|---|---|---|
| age | T0 | T1 | T2 | T0 | T1 | T2 |
| 6.1 | 0.02 | 0.09 | 0.09 | 0.7 | 0.75 | 1.22 |
| 6.3 | 0.15 | 0.38 | 0.46 | 1.52 | 1.70 | 1.74 |
| 7.1 | 0.08 | 0.18 | 0.17 | 0.57 | 0.87 | 1.18 |
| 7.7 | 0.36 | 0.41 | 0.45 | 1.15 | 1.15 | 1.23 |

1. Method for the treatment of spinal muscular atrophy comprising administering a therapeutically effective amount of a therapeutically acceptable salt of phenylbutyrate to a subject in need of treatment of spinal muscular atrophy.

2. Method according to claim 4 where the salt is selected from the group consisting of the sodium, potassium, magnesium, calcium and arginine salts.

3. Method according to claim 4 where the salt is the sodium salt.
